(19) United States
(12) Patent Application Publication
(10) Pub. No.: US 2023/0187213 A1
(43) Pub. Date: Jun. 15, 2023

(54) NANOFABRICATION OF COLLAPSE-FREE HIGH ASPECT RATIO NANOSTRUCTURES

(71) Applicant: Board of Regents, The University of Texas System, Austin, TX (US)

(72) Inventors: Sidlgata V. Sreenivasan, Austin, TX (US); Akhila Mallavarapu, Philadelphia, PA (US); Paras Ajay, Austin, TX (US); Mariana Castaneda, Austin, TX (US); Crystal Barrera, Austin, TX (US)

(21) Appl. No.: 17/923,473

(22) PCT Filed: May 5, 2021
(86) PCT No.: PCT/US2021/030792

§ 371 Date: Nov. 4, 2022

Related U.S. Application Data
(60) Provisional application No. 63/020,408, filed on May 5, 2020.

(51) Int. Cl. H01L 21/306; H01L 21/308

(52) U.S. Cl. CPC H01L 21/30604; H01L 21/3085; H01L 21/3086; B82Y 40/00

(57) ABSTRACT

A method for fabricating silicon nanostructures. An etch uniformity improving layer is deposited on a substrate. A catalyst (e.g., thin film of Ti/Au) is deposited on the substrate or the etch uniformity improving layer, where the catalyst is contacting a portion of the substrate or the etch uniformity layer. The catalyst and the substrate or etch uniformity improving layer are exposed to an etchant, where the catalyst causes etching of the substrate thereby creating etched nanostructures.

### CROSS REFERENCE TO RELATED APPLICATIONS

This application claims priority to U.S. Provisional Pat. Application Serial No. 63/020,408 entitled “Nanofabrication of Collapse-Free High Aspect Ratio Nanostructures,” filed on May 5, 2020, which is incorporated by reference herein in their entirety.

### TECHNICAL FIELD

The present invention relates generally to catalyst influenced chemical etching (CICE), and more particularly to patterning a catalyst and improving large area etch uniformity.

### BACKGROUND

Catalyst Influenced Chemical Etching (CICE) is a catalyst-based etching method that can be used to fabricate features in semiconductors, such as silicon, germanium, etc., where such features have high aspect ratios, low sidewall taper, low sidewall roughness, and/or controllable porosity. Silicon nanostructures made with CICE can enable low-cost, high performance devices for sensors, batteries, thermo-electrics, particle separation arrays and metamaterials.

Large area wafer scale uniform etching of CICE has been demonstrated in literature in creating periodic microscale silicon wires or microscale holes using silver as a catalyst. Nanoscale features have been demonstrated on a wafer with nanosphere lithography and with gold sputtering to get black silicon. However, these processes cannot be easily translated to related patterning and CICE with nanoimprint lithography. Patterning of the catalyst plays a critical role in ensuring large area etch uniformity.

### SUMMARY

In one embodiment of the present invention, a method for fabricating silicon nanostructures comprises depositing an etch uniformity improving layer on a substrate. The method further comprises depositing a catalyst on the substrate or the etch uniformity improving layer, where the catalyst layer is contacting a portion of the substrate or the etch uniformity improving layer. The method additionally comprises exposing the catalyst as well as the substrate or the etch uniformity improving layer to an etchant, where the catalyst causes etching of the substrate thereby creating etched nanostructures.

In another embodiment of the present invention, a method for fabricating silicon nanostructures comprises depositing an etch uniformity improving layer on a substrate. The method further comprises depositing and pattering a resist forming a resist layer with a plurality of features, where the resist layer includes a residual layer of thickness less than 100 nm. The method additionally comprises etching the resist layer to remove the residual layer. Furthermore, the method comprises depositing a catalyst on the substrate or the etch uniformity improving layer, where the catalyst is contacting a portion of the substrate or the etch uniformity improving layer. Additionally, the method comprises exposing the catalyst as well as the substrate or the etch uniformity improving layer to an etchant, where the catalyst causes etching of the substrate thereby creating etched nanostructures.

In a further embodiment of the present invention, a method for fabricating nanostructures of varying heights comprises providing a catalyst layer on a surface of a semiconducting substrate, where the catalyst layer comprises a plurality of features and one or more intentional discontinuities. The method further comprises exposing the catalyst layer on the surface of the semiconducting substrate to an etchant, where the catalyst layer causes etching of the semiconducting substrate starting from the one or more intentional discontinuities, and where fabricated structures have a height variation with features closest to the one or more intentional discontinuities having a maximum height.

In another embodiment of the present invention, a method for fabricating silicon nanostructures comprises patterning a polymer resist on a substrate with a plurality of features. The method further comprises depositing a material conformally on the polymer resist to reduce spacing between the plurality of features. The method additionally comprises providing a catalyst layer on the substrate, where the catalyst layer is patterned using the plurality of features with the reduced spacing such that the catalyst layer contacts only a portion of the substrate. Furthermore, the method comprises exposing the catalyst layer to an etchant, where the catalyst layer causes etching of the substrate thereby creating etched nanostructures.

In a further embodiment of the present invention, a method for fabricating nanostructures in a material comprises etching silicon structures using catalyst influenced chemical etching, where the etched silicon structures are designed to avoid substantial collapse. The method further comprises depositing one or more materials conformally on the etched silicon structures. The method additionally comprises creating access to the etched silicon structures and removing the etched silicon structures selectively leaving the one or more materials substantially the same.

In another embodiment of the present invention, a method for fabricating nanostructures in a silicon layer on a non-silicon layer comprises etching nanostructures in silicon using metal assisted chemical etching, where the etched nanostructures are designed to avoid substantial collapse. The method further comprises partially or completely oxidizing the etched nanostructures.

In a further embodiment of the present invention, nanostructures in a silicon layer on a non-silicon layer that possess optical lensing properties, where a core geometry is first etched into the silicon layer while substantially avoiding collapse, and where the core geometry is subsequently oxidized partially or fully.

In another embodiment of the present invention, a device using silicon nanostructures comprises silicon nanostructures designed to separate particles in a fluid medium having different size, shape or flow properties in a nanostructure array, where spacing between at least a pair of silicon nanostructures is less than 50 nm, and where a nanostructure wall angle of one or more of the silicon nanostructures is greater than 89.5 degrees at all points on a side wall except for a top and a bottom of the side wall.

In a further embodiment of the present invention, a device for separation and detection of biological species comprises silicon nanostructures fabricated using catalyst influenced chemical etching, where the silicon nanostructures are designed for particle separation in a fluid medium. The device further comprises sensors which are used to detect target species in the separated particles, where the sensors generate electrical and/or optical signals based on desired target species detection.

The foregoing has outlined rather generally the features and technical advantages of one or more embodiments of the present invention in order that the detailed description of the present invention that follows may be better understood. Additional features and advantages of the present invention will be described hereinafter which may form the subject of the claims of the present invention.

### BRIEF DESCRIPTION OF THE DRAWINGS

A better understanding of the present invention can be obtained when the following detailed description is considered in conjunction with the following drawings, in which:

FIG. 1 is a flowchart of a method for patterning of catalysts after imprint in accordance with an embodiment of the present invention;

FIGS. 2A-2G depict cross-sectional views for patterning of catalysts after imprint using the steps described in FIG. 1 in accordance with an embodiment of the present invention;

FIG. 3 illustrates a process flow for uniform CICE with an underlying oxide layer and undercut as described in the method of FIG. 1 in accordance with an embodiment of the present invention;

FIG. 4 is a flowchart of an alternative method for patterning of catalysts after imprint in accordance with an embodiment of the present invention;

FIGS. 5A-5H depict cross-sectional views for patterning of catalysts after imprint using the steps described in FIG. 4 in accordance with an embodiment of the present invention;

FIG. 6 is a flowchart of a further alternative method for patterning of catalysts after imprint in accordance with an embodiment of the present invention;

FIGS. 7A-7F depict cross-sectional views for patterning of catalysts after imprint using the steps described in FIG. 6 in accordance with an embodiment of the present invention;

FIG. 8 is a flowchart of an additional alternative method for patterning of catalysts after imprint in accordance with an embodiment of the present invention;

FIGS. 9A-9E depict cross-sectional views for patterning of catalysts after imprint using the steps described in FIG. 8 in accordance with an embodiment of the present invention;

FIGS. 10A-10B illustrate the effect of continuous versus discontinuous catalysis on CICE etch variation in accordance with an embodiment of the present invention;

FIGS. 11A-11C illustrate analog etch depth variation in CICE using pinholes in the catalyst film in accordance with an embodiment of the present invention;

FIG. 12 illustrates the process steps to vary the diameter of an imprinted resist pattern to fabricate silicon nanowires with precisely controlled feature dimensions at a constant pitch in accordance with an embodiment of the present invention;

FIG. 13 is a flowchart of a method for a conformal deposition process to obtain desired material high aspect ratio (HAR) nanostructures with variants of the nanostructure geometry using no replacement steps in accordance with an embodiment of the present invention;

FIGS. 14A-14D depict cross-sectional views for obtaining HAR nanostructures using the steps described in FIG. 13 in accordance with an embodiment of the present invention;

FIG. 15A, 15B1, 15B2 and 15C depict the variants of the nanostructure geometry using the conformal deposition process of FIG. 13 in accordance with an embodiment of the present invention;

FIGS. 16A-16C illustrate different variants of the nanostructure geometry using the conformal deposition process of FIG. 13 in accordance with an embodiment of the present invention;

FIG. 17 is a method for obtaining the desired material high aspect ratio (HAR) nanostructures using a replacement process and atomic layer deposition (ALD) in accordance with an embodiment of the present invention;

FIGS. 18A-18E depict cross-sectional views for obtaining the desired HAR nanostructures using the steps described in FIG. 17 in accordance with an embodiment of the present invention;

FIG. 19 is a flowchart of a method for obtaining the desired material high aspect ratio (HAR) nanostructures using a replacement process and atomic layer deposition (ALD) after exfoliation of silicon in accordance with an embodiment of the present invention;

FIGS. 20A-20E depict cross-sectional views for obtaining the desired HAR nanostructures using the steps described in FIG. 19 in accordance with an embodiment of the present invention;

FIG. 21 is a flowchart of a method for achieving nanostructures in the desired material in accordance with an embodiment of the present invention;

FIGS. 22A-22D depict cross-sectional views achieving nanostructures in the desired material using the steps described in FIG. 21 in accordance with an embodiment of the present invention; and

FIG. 23 illustrates silicon nanopillars made with CICE for DLD-based particle separation in accordance with an embodiment of the present invention.

### DETAILED DESCRIPTION

As stated in the Background section, large area wafer scale uniform etching of catalyst influenced chemical etching (CICE) has been demonstrated in literature in creating periodic microscale silicon wires or microscale holes using silver as a catalyst. Nanoscale features have been demonstrated on a wafer with nanosphere lithography and with gold sputtering to get black silicon. However, these processes cannot be easily translated to related patterning and CICE with nanoimprint lithography. Patterning of the catalyst plays a critical role in ensuring large area etch uniformity.

The principles of the present invention provide a means for patterning a catalyst and improving large area etch uniformity as well as providing intentional etch variation and control. Furthermore, embodiments of the present invention are used to pattern silicon nanostructures with very high aspect ratios. Additionally, embodiments of the present invention are used to pattern non-silicon nanostructures by post-processing after CICE which can enable applications with high aspect ratio metal/semiconductor/insulator/transparent nanostructures. Packaging of the fabricated devices is also described herein.

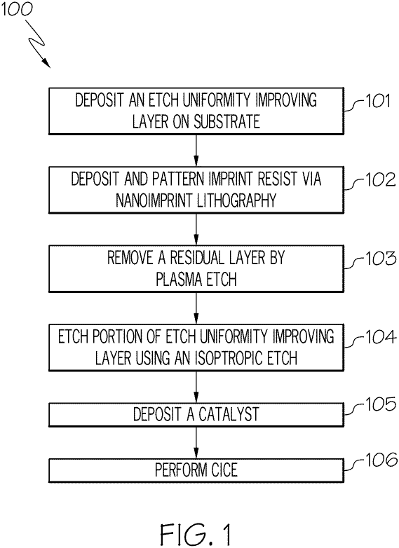

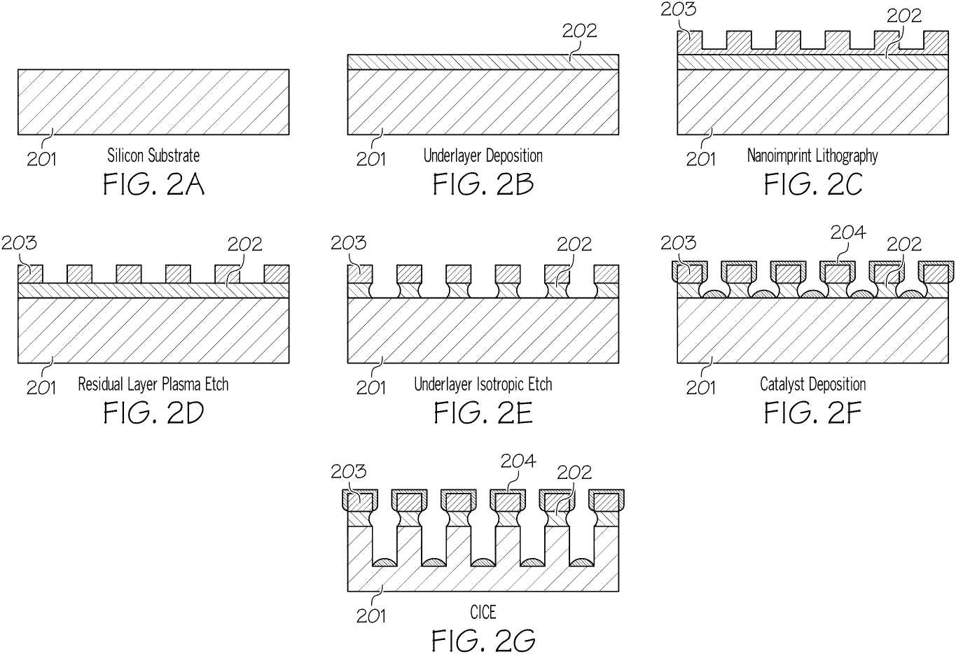

Referring now to the Figures in detail, FIG. 1 is a flowchart of a method 100 for patterning of catalysts after imprint in accordance with an embodiment of the present invention. FIGS. 2A-2G depict cross-sectional views for patterning of catalysts after imprint using the steps described in FIG. 1 in accordance with an embodiment of the present invention.

Referring to FIG. 1, in conjunction with FIGS. 2A-2G, in step 101, a deposition (e.g., underlying deposition) of an etch uniformity improving layer (e.g., silicon oxide) 202 on substrate 201 (e.g., silicon substrate) is performed, as shown in FIGS. 2A and 2B. In one embodiment, the thickness of etch uniformity improving layer 202 ranges between 5 nm and 100 nm. In one embodiment, etch uniformity improving layer 202 is grown thermally on substrate 201 (e.g., crystalline silicon substrate). In one embodiment, etch uniformity improving layer 202 is a native silicon oxide layer.

In step 102, an imprint resist 203 (e.g., monomer or polymer formulation) is deposited and patterned (forming nanostructures) on etch uniformity improving layer 202 via nanoimprint lithography as shown in FIG. 2C.

In step 103, a residual layer (residual of imprint resist 203) is removed by plasma etch, such as between the nanostructures, as shown in FIG. 2D.

In step 104, a portion of etch uniformity improving layer 202 is etched, including between and underneath the nanostructures, such as via an undercut, as shown in FIG. 2E using an isotropic etch. In one embodiment, the etchant includes two or more of the following: fluoride species containing chemicals HF or NH₄F, oxidants (e.g., H₂O₂, KMnO₄), alcohols (e.g., ethanol, isopropyl alcohol, ethylene glycol) and solvents (e.g., protic, aprotic, polar and nonpolar solvents).

In step 105, a catalyst 204 is deposited, such as over and between the nanostructures, as shown in FIG. 2F. In one embodiment, catalyst 204 is a thin film of Ti/Au.

In step 106, CICE is performed as shown in FIG. 2G. In one embodiment, the portion of substrate 201 underneath catalyst 204 is etched in the CICE solution whereby catalyst 204 proceeds to etch into substrate 201. In one embodiment, the resulting structure includes a nanostructure array (etched nanostructures) designed to separate particles in a fluid medium having different size, shape or flow properties, where a spacing in the etched nanostructures is designed to separate said particles. In one embodiment, a geometry of individual pillars of said nanostructure array is determined by flow profiles. In one embodiment, the geometry of individual pillars of the nanostructure array is optimized for its shape, where the shape includes one of the following: circular, triangular, square, rhombic, and air-foil. In one embodiment, the individual pillars of the nanostructure array are capped to confine a flow of particles to between gaps in the nanostructure array.

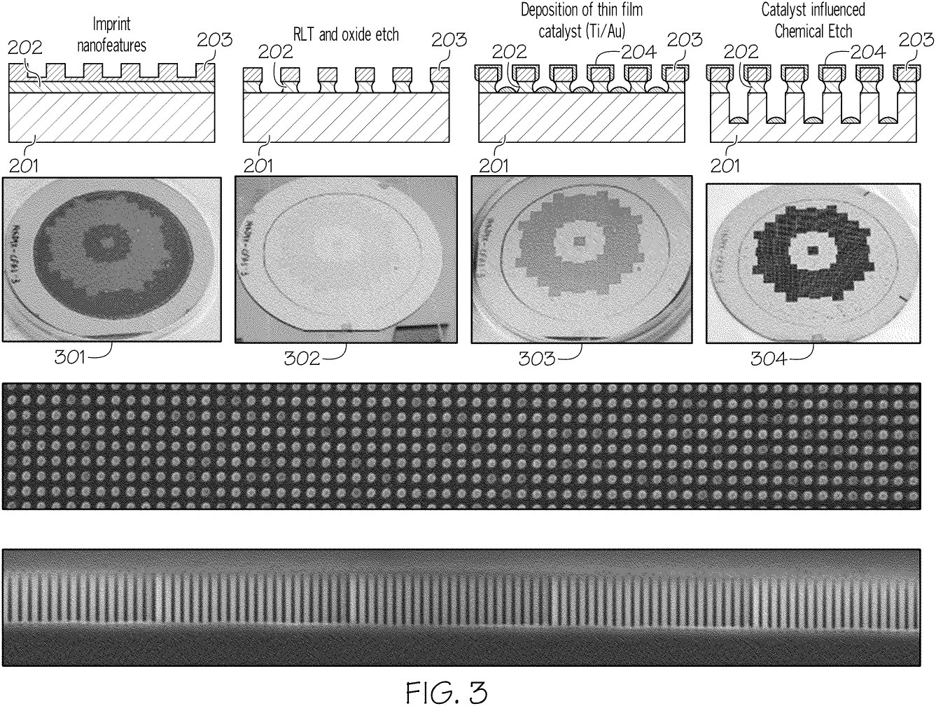

FIG. 3 illustrates a process flow for uniform CICE with an underlying oxide layer and undercut as described in method 100 in accordance with an embodiment of the present invention.

Referring to FIG. 3, in conjunction with FIGS. 1 and 2A-2G, FIG. 3 illustrates the imprint nanofeatures 301 formed by patterning imprint resist 203 using nanoimprint lithography as shown in FIG. 2C.

As discussed above, the residual layer thickness and etch uniformity improving layer 202 (e.g., silicon oxide) are etched as illustrated in image 302 and shown in FIG. 2E.

Furthermore, as discussed above, a thin film catalyst 204, such as Ti/Au, is deposited, such as between the nanostructures, as shown in FIG. 2F and in image 303.

Lastly, as discussed above, a CICE is performed in which a portion of substrate 201 underneath catalyst 204 is etched in the CICE solution whereby catalyst 204 proceeds to etch into substrate 201 as shown in FIG. 2G and in image 304.

In one embodiment, FIG. 3 illustrates the experimental results of method 100 in which an underlayer between the resist and silicon is used to improve etch uniformity by inducing etch uniformity by (a) creating an undercut, and/or (b) enhancing etchant transport due to etching in the CICE solution and/or improving wetting of etchants thereby enabling a uniform “starting point” throughout the wafer. This ensures that the etch starts at the same point in all portions of the wafer and ensures etch depth uniformity.

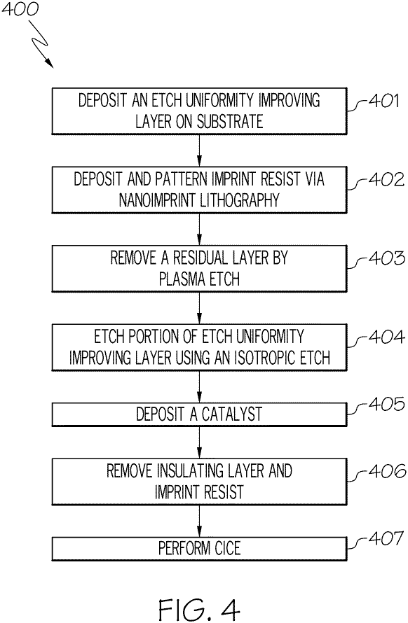

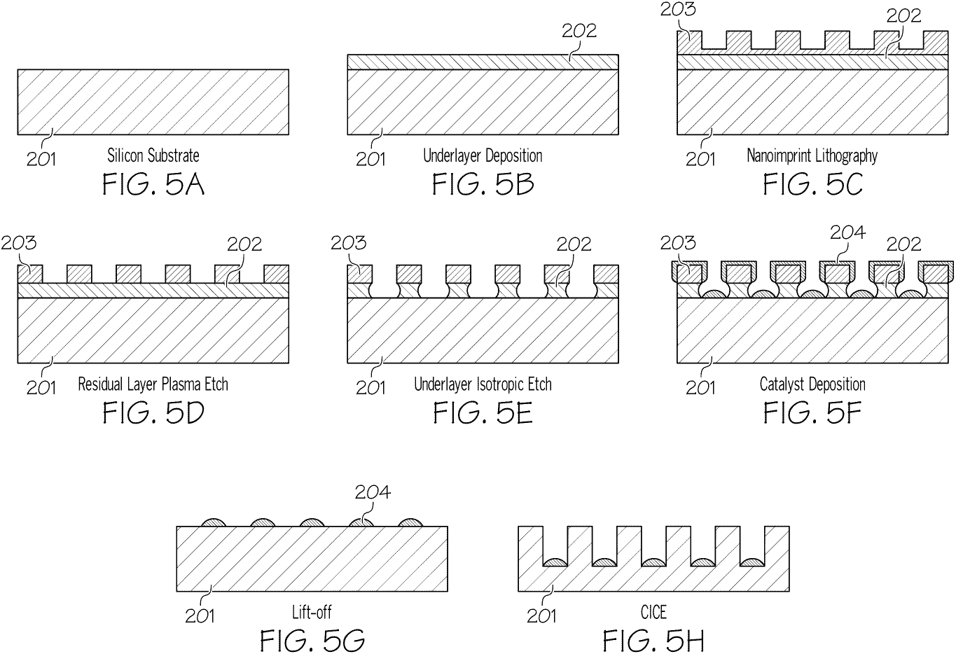

Referring now to FIG. 4, FIG. 4 is a flowchart of an alternative method 400 for patterning of catalysts after imprint in accordance with an embodiment of the present invention. FIGS. 5A-5H depict cross-sectional views for patterning of catalysts after imprint using the steps described in FIG. 4 in accordance with an embodiment of the present invention.

Referring to FIG. 4, in conjunction with FIGS. 5A-5H, in step 401, a deposition (e.g., underlying deposition) of an insulating layer (e.g., silicon oxide) 202 on substrate 201 (e.g., silicon substrate) is performed, as shown in FIGS. 5A and 5B. In one embodiment, the thickness of etch uniformity improving layer 202 ranges between 5 nm and 100 nm. In one embodiment, etch uniformity improving layer 202 is grown thermally on substrate 201 (e.g., crystalline silicon substrate). In one embodiment, etch uniformity improving layer 202 is a native silicon oxide layer.

In step 402, an imprint resist 203 (e.g., monomer or polymer formulation) is deposited and patterned (forming nanostructures) on etch uniformity improving layer 202 via nanoimprint lithography as shown in FIG. 5C.

In step 403, a residual layer (residual of imprint resist 203) is removed by plasma etch, such as between the nanostructures formed by imprint resist 203, as shown in FIG. 5D.

In step 404, a portion of etch uniformity improving layer 202 is etched, including between and underneath the nanostructures, such as via an undercut, as shown in FIG. 5E using an isotropic etch. In one embodiment, the etchant includes two or more of the following: fluoride species containing chemicals HF or NH4F, oxidants (e.g., H₂O₂, KMnO₄), alcohols (e.g., ethanol, isopropyl alcohol, ethylene glycol) and solvents (e.g., protic, aprotic, polar and nonpolar solvents).

In step 405, a catalyst 204 is deposited, such as over and between the nanostructures, as shown in FIG. 5F. In one embodiment, catalyst 204 is a thin film of Ti/Au.

In step 406, etch uniformity improving layer 202 and imprint resist 203 are removed, such as via a lift-off process, as shown in FIG. 5G.

In step 407, CICE is performed as shown in FIG. 5H. In one embodiment, the portion of substrate 201 underneath catalyst 204 is etched in the CICE solution whereby catalyst 204 proceeds to etch into substrate 201. In one embodiment, the resulting structure includes a nanostructure array (etched nanostructures) designed to separate particles in a fluid medium having different size, shape or flow properties, where a spacing in the etched nanostructures is designed to separate said particles. In one embodiment, a geometry of individual pillars of said nanostructure array is determined by flow profiles. In one embodiment, the geometry of individual pillars of the nanostructure array is optimized for its shape, where the shape includes one of the following: circular, triangular, square, rhombic, and air-foil. In one embodiment, the individual pillars of the nanostructure array are capped to confine a flow of particles to between gaps in the nanostructure array.

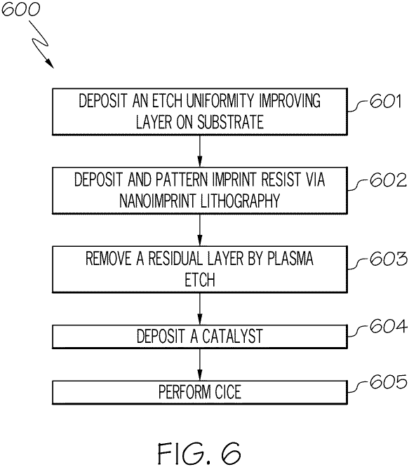

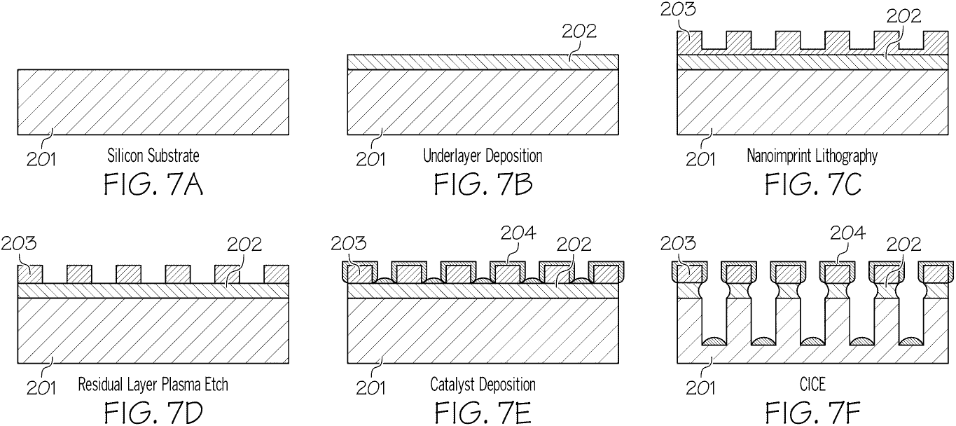

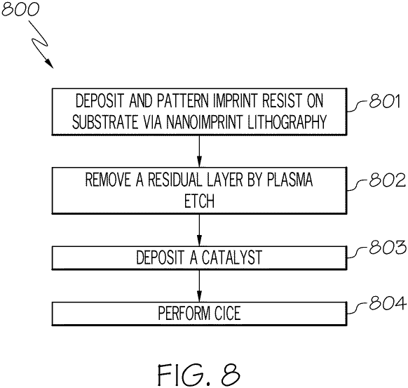

Referring now to FIG. 6, FIG. 6 is a flowchart of a further alternative method 600 for patterning of catalysts after imprint in accordance with an embodiment of the present invention. FIGS. 7A-7F depict cross-sectional views for patterning of catalysts after imprint using the steps described in FIG. 6 in accordance with an embodiment of the present invention.

Referring to FIG. 6, in conjunction with FIGS. 7A-7F, in step 601, a deposition (e.g., underlying deposition) of an insulating layer (e.g., silicon oxide) 202 on substrate 201 (e.g., silicon substrate) is performed, as shown in FIGS. 7A and 7B. In one embodiment, the thickness of etch uniformity improving layer 202 ranges between 5 nm and 100 nm. In one embodiment, etch uniformity improving layer 202 is grown thermally on substrate 201 (e.g., crystalline silicon substrate). In one embodiment, etch uniformity improving layer 202 is a native silicon oxide layer.

In step 602, an imprint resist 203 (e.g., monomer or polymer formulation) is deposited and patterned (forming nanostructures) on etch uniformity improving layer 202 via nanoimprint lithography as shown in FIG. 7C.

In step 603, a residual layer (residual of imprint resist 203) is removed by plasma etch, such as between the nanostructures formed by imprint resist 203, as shown in FIG. 7D.

In step 604, a catalyst 204 is deposited, such as over and between the nanostructures, as shown in FIG. 7E. In one embodiment, catalyst 204 is a thin film of Ti/Au.

In step 605, CICE is performed as shown in FIG. 7F. In one embodiment, the portion of substrate 201 underneath catalyst 204 is etched in the CICE solution whereby catalyst 204 proceeds to etch into substrate 201. In one embodiment, the resulting structure includes a nanostructure array (etched nanostructures) designed to separate particles in a fluid medium having different size, shape or flow properties, where a spacing in the etched nanostructures is designed to separate said particles. In one embodiment, a geometry of individual pillars of said nanostructure array is determined by flow profiles. In one embodiment, the geometry of individual pillars of the nanostructure array is optimized for its shape, where the shape includes one of the following: circular, triangular, square, rhombic, and air-foil. In one embodiment, the individual pillars of the nanostructure array are capped to confine a flow of particles to between gaps in the nanostructure array.

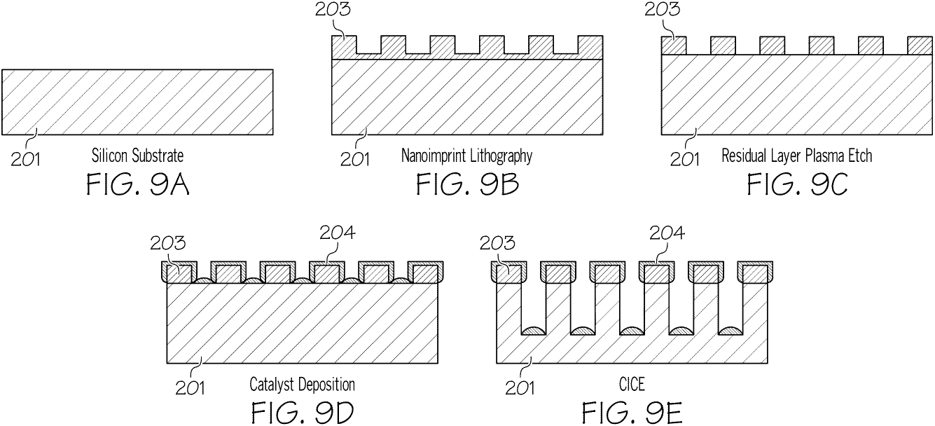

Referring now to FIG. 8, FIG. 8 is a flowchart of an additional alternative method 800 for patterning of catalysts after imprint in accordance with an embodiment of the present invention. FIGS. 9A-9E depict cross-sectional views for patterning of catalysts after imprint using the steps described in FIG. 8 in accordance with an embodiment of the present invention.

Referring to FIG. 8, in conjunction with FIGS. 9A-9E, in step 801, an imprint resist 203 (e.g., monomer or polymer formulation) is deposited and patterned (forming nanostructures) on substrate 201 via nanoimprint lithography as shown in FIGS. 9A and 9B.

In step 802, a residual layer (residual of imprint resist 203) is removed by plasma etch, such as between the nanostructures formed by imprint resist 203, as shown in FIG. 9C.

In step 803, a catalyst 204 is deposited, such as over and between the nanostructures, as shown in FIG. 9D. In one embodiment, catalyst 204 is a thin film of Ti/Au.

In step 804, CICE is performed as shown in FIG. 9E. In one embodiment, the portion of substrate 201 underneath catalyst 204 is etched in the CICE solution whereby catalyst 204 proceeds to etch into substrate 201. In one embodiment, the resulting structure includes a nanostructure array (etched nanostructures) designed to separate particles in a fluid medium having different size, shape or flow properties, where a spacing in the etched nanostructures is designed to separate said particles. In one embodiment, a geometry of individual pillars of said nanostructure array is determined by flow profiles. In one embodiment, the geometry of individual pillars of the nanostructure array is optimized for its shape, where the shape includes one of the following: circular, triangular, square, rhombic, and air-foil. In one embodiment, the individual pillars of the nanostructure array are capped to confine a flow of particles to between gaps in the nanostructure array.

As discussed above, FIGS. 1, 2A-2G, 3, 4, 5A-5H, 6, 7A-7F, 8 and 9A-9E describe five processes for patterning of catalysts for CICE.

In one embodiment, catalyst 204 includes one or more of the following: Au, Pt, Pd, Mo, Ir, Ru, Ag, Cu, Ni, W, TiN, TaN, RuO₂, IrO₂, graphene, Ti, and carbon. In one embodiment, catalyst 204 has an adhesion layer. In one embodiment, catalyst 204 is gold and the adhesion layer is Ti. In another embodiment, catalyst 204 is Ru and the adhesion layer is Ti.

In one embodiment, catalyst 204 is patterned using one of the following: nanoimprint lithography, photolithography, focused ion beam milling, electron beam lithography, laser interference lithography, nanosphere lithography, block copolymer lithography, and directed self-assembly.

In one embodiment, the features fabricated using CICE have a critical dimension of less than 200 nm, a height of more than 200 nm, and a wall taper angle greater than 89.5 degrees. In one embodiment, the wall taper angle at any point along the sidewall is greater than 89.5 degrees. In one embodiment, the taper angle is 89.9 degrees. In another embodiment, the angle is 90 degrees. In one embodiment, the points along the sidewall do not include the top-most point and bottom-most point (where the angle changes from zero degrees for the horizontal plane to 90 degrees at the sidewall plane).

In one embodiment, the aspect ratio of the features is greater than 5. In another embodiment, the aspect ratio is greater than 10. In another embodiment, the aspect ratio is greater than 20. In one embodiment, the aspect ratio is greater than 100.

In one embodiment, the nanofeatures have shaped cross-sections with sharp corners having a radius of curvature less than 10 nm. In another embodiment, the radius of curvature is less than 5 nm. In another embodiment, the radius of curvature of a sharp corner is less than 20 nm. Shaped cross-section geometries include diamond, triangle, fractal, square, quadrilateral, star-shaped, bow-tie, airfoil, oval, spiral, etc. Lithography to make such structures is described in U.S. Pat. No. 10,026,609, which is incorporated by referenced herein in its entirety. In one embodiment, e-beam and optical lithography are used. In another embodiment, a multiple patterning technique (e.g., triple, quad pattering, Litho-Etch-Litho-Etch, spacer techniques, etc.) is used to make the features or the template for imprint lithography.

In one embodiment, the features are patterned using nanoimprint lithography. In one embodiment, the residual layer thickness (RLT) of resist after patterning using nanoimprint lithography is less than 50 nm. In one embodiment, the RLT is less than 100 nm. In another embodiment, the RLT is less than 20 nm. In another embodiment, the RLT is less than 10 nm.

In one embodiment, substrate 201 for CICE is a silicon wafer. In another embodiment, substrate 201 is a silicon-on-nonsilicon wafer, such as SOI wafers, silicon-on-sapphire, silicon-on-polymer, silicon-on-metal etc. In one embodiment, substrate 201 is one of the following: single crystal bulk silicon wafer, a layer of polysilicon of thickness greater than 100 nm deposited on a substrate, a layer of amorphous silicon of thickness greater than 100 nm deposited on a substrate, an SOI (silicon on insulator) wafer, silicon-on-glass, silicon-on-sapphire, epitaxial silicon of thickness greater than 100 nm on a substrate, alternating layers of semiconductor materials of varying doping levels and dopants, highly doped silicon and lightly doped silicon, undoped silicon and doped silicon or germanium, silicon and SiₓGe₁₋ₓ, differently doped silicon and/or SiₓGe₁₋ₓ, differently doped silicon and/or Ge, or Si and Ge.

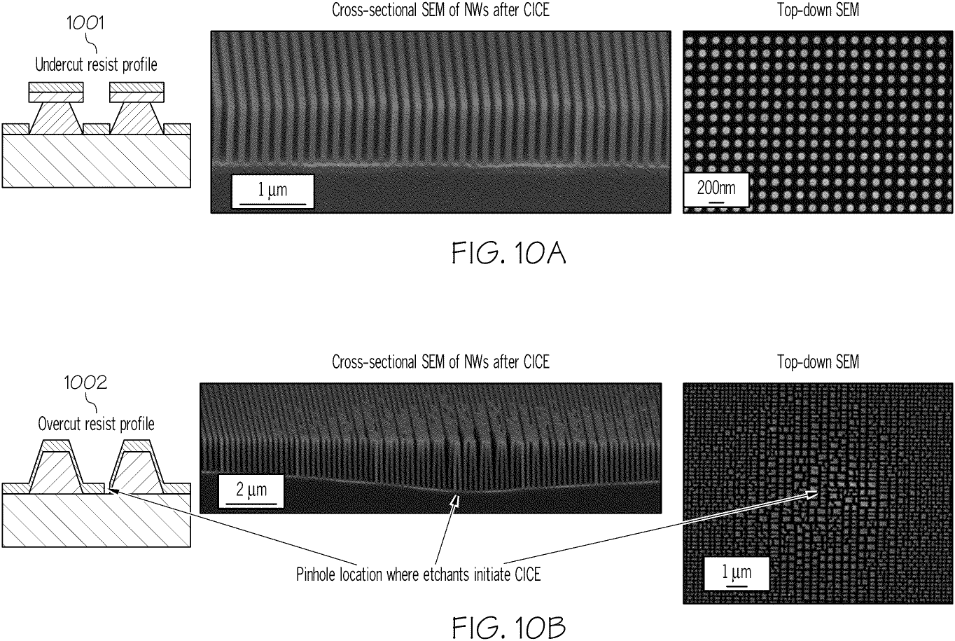

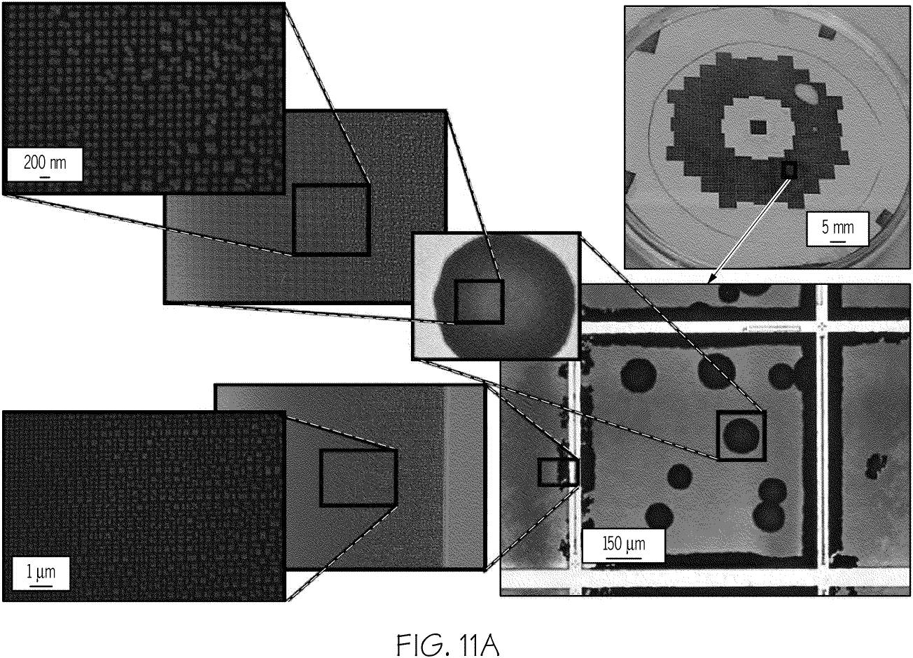

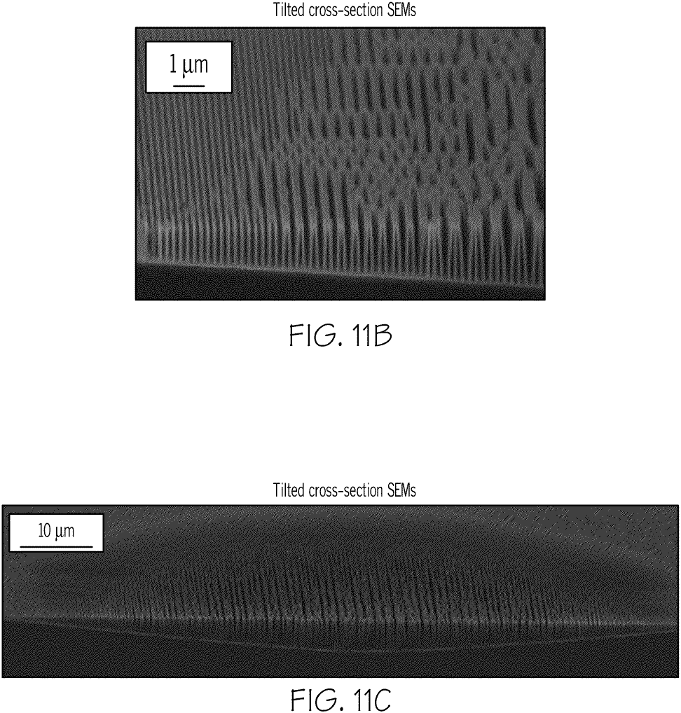

Furthermore, the embodiments of the present invention may perform intentional etch variations using analog CICE. Uniformity of the etch is highly dependent on the resist shape and the thickness of the catalyst. Tuning these parameters can enable intentional analog variation of the etch depth to visualize collapse behavior at the nanoscale. Etchant transport to the metal/silicon interface is critical for uniform MAC-Etch. Etch uniformity is highly dependent on the method of catalyst patterning and the thickness of the film used. In one embodiment, gold patterning is performed using liftoff. Liftoff processes require a break in the gold film after deposition on resist features, where the resist features have an “undercut” profile. Gold on top of the resist features is removed during a wet etch of the resist leaving behind patterned gold on a silicon wafer. Alternatively, as long as there is a break in the gold film, CICE can occur without a liftoff step. For “overcut” resist features, or for thicker films of gold, if a uniform continuous film is deposited on patterned resist without metal breaks, then CICE will start to occur at pinhole defects and discontinuities in catalyst metal on the wafer. The initiation of such pinholes will further enable etchant transport laterally, causing delayed CICE in the surrounding areas, thereby creating nanowires with an analog variation of heights. In one embodiment, the discontinuities are created using one or more the following: focused ion beam, photolithography, imprint lithography, laser writing, and pattern geometries. In one embodiment, the shape of the discontinuities includes one or more of the following: a circular pinhole, a line and a series of intersecting lines. FIGS. 10A-10B illustrate the difference between CICE for gold deposited on “undercut” features, where an oxide underlying layer is used to create an undercut for metal break, compared to “overcut” features with no underlying layer to create a metal break in the nanoscale pattern. The CICE of the two patterns shows the difference in uniformity of the etch as well as the formation of “pinhole-locations” where the CICE process starts.

Referring now to FIGS. 10A-10B, FIGS. 10A-10B illustrate the effect of continuous versus discontinuous catalysis on CICE etch variation in accordance with an embodiment of the present invention. FIG. 10A illustrates the undercut resist profile 1001, whereas, FIG. 10B illustrates the overcut resist profile 1002. FIGS. 10A-10B illustrate the profiles (profiles 1001, 1002, respectively) effect on subsequent CICE to create nanowires.

In one embodiment, the overcut process is used to locate regions with varying nanowire heights, where the onset of collapse can be visualized as the height at which the tips of two or more nanowires start to touch. FIGS. 11A-11C illustrate analog etch depth variation in CICE using pinholes in the catalyst film in accordance with an embodiment of the present invention. In particular, FIGS. 11A-11C illustrate a 100 mm silicon wafer with circular regions showing etch depth variation, which manifest as collapse of tall nanowires. As illustrated in FIGS. 11A-11C, the top-down SEMs show the collapse of nanowires.

In one embodiment, analog CICE is used to intentionally vary etch depths to detect the critical aspect ratio for onset of collapse. In one embodiment, silicon nanowires with varying diameters and etch depths are made using nanoimprint lithography and analog-CICE. The onset of collapse can be found using defect detection algorithms, such as local binary pattern (LBP). At larger diameters (smaller spacing), NW (nanowire) heights for critical collapse are greater than at smaller diameters (larger spacing) at the same pitch. In one embodiment, a combination of increased diameters and associated increased heights observed experimentally, leads to significant enhancements in surface area of the Si NWs.

Embodiments of the present invention enable feature size control for sub-lithographic spacing for CICE.

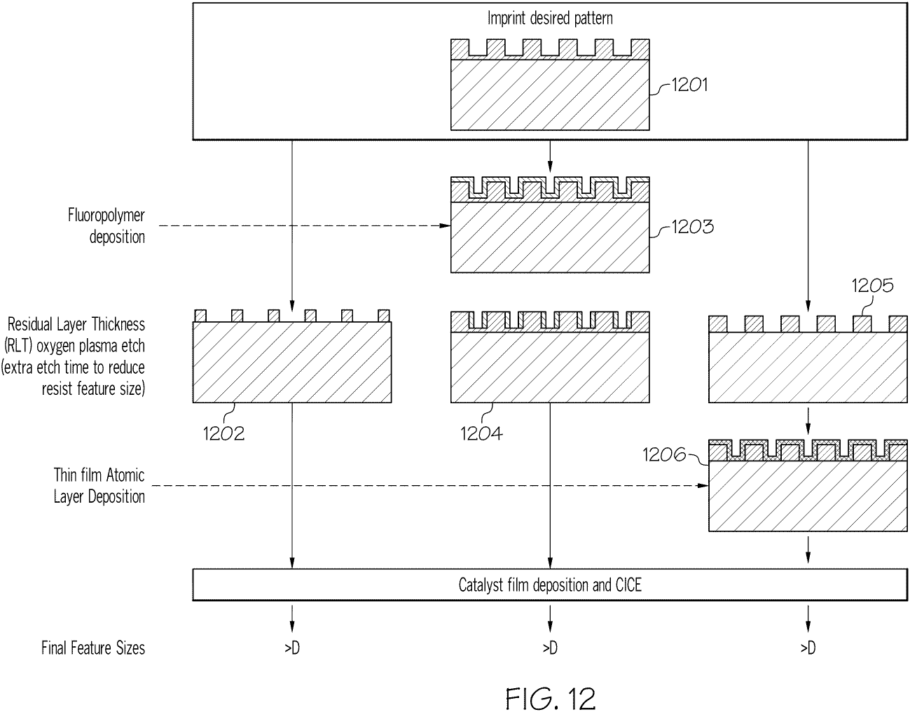

In one embodiment, imprint lithography is used to pattern circular resist pillars with a diameter of 120 nm at a pitch of 200 nm using a template made using electron beam lithography. Varying the diameters of these wires can be done by imprinting with templates having patterns with different diameters. This is, however, very expensive due to the cost of making a template and the long e-beam write times. For a given pitch, plasma etching, chemical vapor deposition, or atomic layer deposition can be used to vary the diameters of the resist after imprinting, prior to gold deposition and CICE. FIG. 12 illustrates the process modification from a typical CICE process to change the diameter of circular nanowires (NW) at a constant pitch.

Referring to FIG. 12, FIG. 12 illustrates the process steps to vary the diameter of an imprinted resist pattern 1201 to fabricate silicon nanowires with precisely controlled feature dimensions at a constant pitch in accordance with an embodiment of the present invention.

In one embodiment, NW diameters ranging from 75-110 nm are obtained by the standard process shown in FIGS. 1 and 2A-2G, with an increased residual layer thickness etch time (see element 1202). This is done using an oxygen and argon plasma, with a vertical etch rate of 30 nm/min and a lateral etch rate of 5 nm/min. Varying the etch times to simultaneously remove the RLT and reduce the diameter enables a reduction in nanowire diameters.

In one embodiment, for NW diameters ranging from 110 nm - 140 nm, a chemical vapor deposition (CVD) process is used to deposit fluoropolymer (see element 1203) on the imprinted resist 1201 by flowing C₄F₈ gas in a plasma reactor. A thin conformal layer of fluoropolymer is deposited increasing the diameter of the resist. Varying the RLT etch times (see element 1204) is used to remove the RLT and reduce the diameter.

In one embodiment, for diameters ranging from 140-175 nm, a conformal layer of a film (that can be etched in the CICE solution, e.g., silicon oxide, aluminum oxide) is deposited using atomic layer deposition (ALD) (see element 1206), after the imprint and RLT etch (see element 1205). In one embodiment, 30 nm of aluminum oxide is deposited on a resist pillar of diameter 110 nm (after the RLT etch) which results in forming a pillar with a diameter of 170 nm. Gold deposition and CICE results in silicon nanowires with a diameter of 170 nm. In one embodiment, the thickness of the wires can be varied by changing the ALD film thickness. The ALD oxide gets etched away during the CICE process.

The following discusses nanostructures that utilize CICE.

CICE is used to make high aspect ratio (HAR) Si nanostructures of arbitrary geometries. In one embodiment, these structures are made in silicon on a non-silicon substrate. In one embodiment, the silicon is single-crystal silicon, and the non-silicon substrate is silicon oxide, sapphire, a polymer, such as polycarbonate, metal, such as hastealloy, etc.

In one embodiment, silicon nanostructures made using CICE are oxidized to convert them partially or substantially into silicon oxide. In one embodiment, the silicon is oxidized before deposition of the desired material using thermal oxidation, plasma oxidation, anodic oxidation, light-based (e.g., vacuum ultraviolet (VUV)) oxidation, ozone-based oxidation, etc.

The geometry of the silicon pillars to be etched by CICE (and subsequently oxidized) is optimized to take into consideration the geometry of the pillars to minimize collapse and change in feature size due to oxidation.

In one embodiment, material is deposited on silicon nanostructures etched using CICE using conformal deposition methods, such as atomic layer deposition (ALD), chemical vapor deposition (CVD), physical vapor deposition, thermal oxidation, electrodeposition, etc. In one embodiment, the deposited (shell) material is TiO₂. In another embodiment, the deposited material is SiO₂. In another embodiment, no material is deposited, and the silicon is oxidized all the way. In another embodiment, silicon nanostructures are oxidized to less than 10% of their volume. In another embodiment, silicon nanostructures are oxidized to less than 50% of their volume.

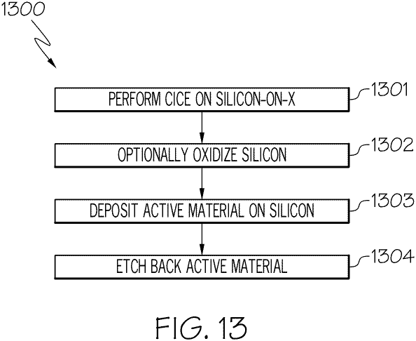

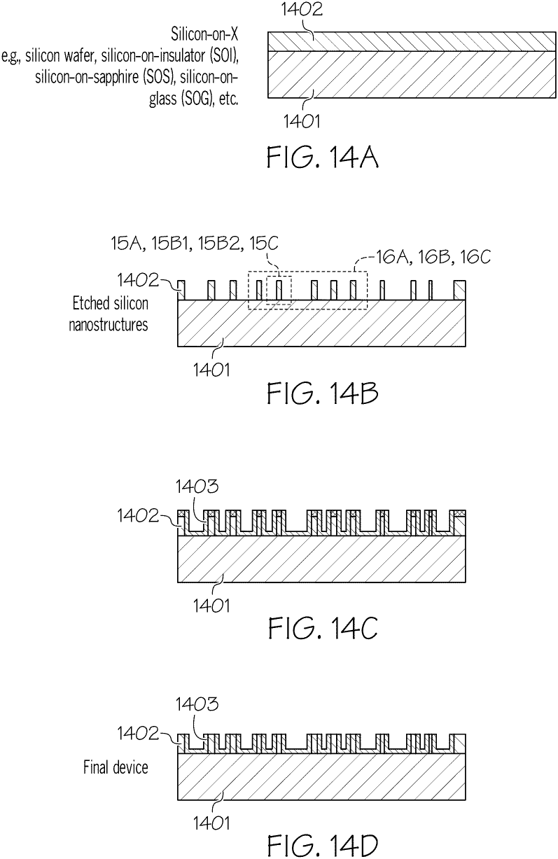

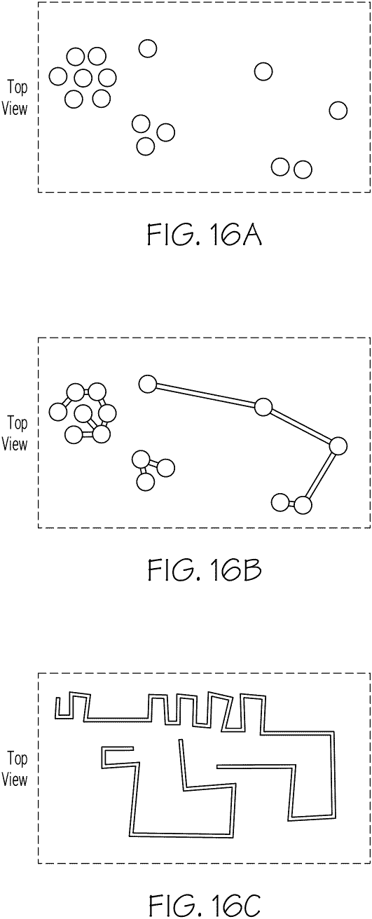

In another embodiment, the silicon nanostructures (or cores) have a space filling geometry. In another embodiment, the silicon nanostructures (or cores) have a high degree of rotational symmetry. In one embodiment, the high degree of rotational symmetry refers to the core cross-section having a rotational symmetry of order 6 or more. In another embodiment, the silicon nanostructures are axisymmetric (FIGS. 13, 14A-14D, 15A, 15B1, 15B2 and 15C). In another embodiment, the silicon nanostructures have varying pitch to modulate the local packing density of the nanostructures. In another embodiment, the silicon structures are linked together using a maze, which are in the shape of a group of acyclic undirected graphs. In another embodiment, only silicon structures consisting of the acyclic undirected maze are present (FIGS. 14A-14D and 16A-16C).

In another embodiment, the nanostructures are made using porous silicon (which can be generated using the CICE process), which is subsequently oxidized.

In the case where the nanostructures are used for lensing applications (such as metalenses), the core, or the shell, or both, are doped, either prior to CICE or post CICE, to modify their refractive indices. In another embodiment, nanostructures have appropriate roughness to reduce light loss due to reflection at material interfaces (Air-Si, SiO₂—TiO₂, etc.). In another embodiment, the nanostructures are coated with an anti-reflective coating to reduce light loss cure to reflection at material interfaces.

In one embodiment, the core structures have a minimum feature size less than or equal to 50 nm. In another embodiment, the core structures have a minimum feature size less than or equal to 100 nm. In another embodiment, the core structures have a minimum feature size less than or equal to 200 nm. In one embodiment, a surface of the core structures has roughness to reduce interfacial reflective losses. In one embodiment, the core structures are coated with an anti-reflective coating.

In one embodiment, the shell structures have a thickness larger than 10 nm. In another embodiment, the shell structures have a thickness of larger than 50 nm. In another embodiment, the shell structures have a thickness of larger than 100 nm. In one embodiment, a surface of the shell structures has roughness to reduce interfacial reflective losses. In one embodiment, the shell structures are coated with an anti-reflective coating.

In one embodiment, the core structures have a height larger than 100 nm. In another embodiment, the core structures have a height larger than 500 nm. In one embodiment, the core structures have a height larger than 1 µm. In another embodiment, the core structures have a height larger than 2 µm.

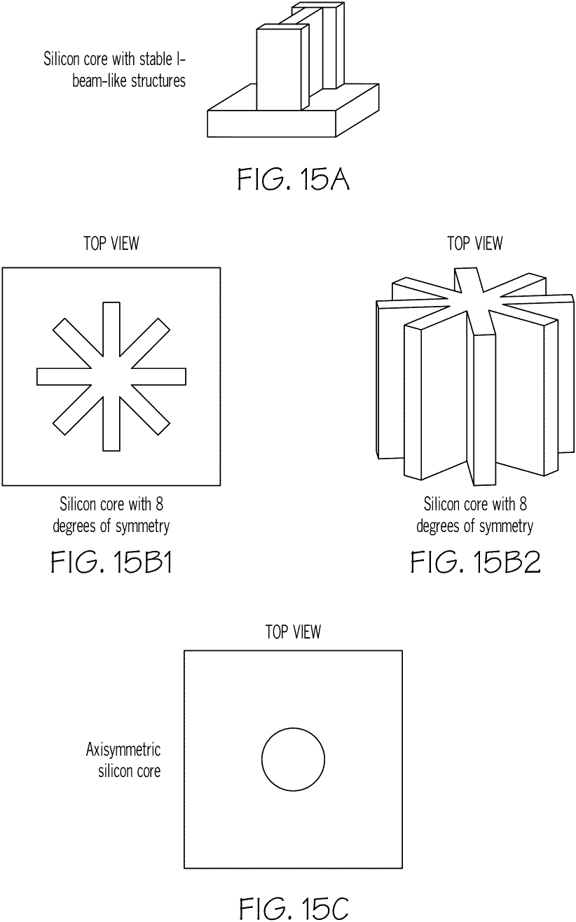

Referring now to FIG. 13, FIG. 13 is a flowchart of a method 1300 for a conformal deposition process to obtain desired material high aspect ratio (HAR) nanostructures with variants of the nanostructure geometry using no replacement steps in accordance with an embodiment of the present invention. FIGS. 14A-14D depict cross-sectional views for obtaining HAR nanostructures using the steps described in FIG. 13 in accordance with an embodiment of the present invention. FIG. 15A, 15B1, 15B2 and 15C depict the variants of the nanostructure geometry using the conformal deposition process of FIG. 13 in accordance with an embodiment of the present invention.

Referring to FIG. 13, in conjunction with FIGS. 14A-14D and 15A, 15B1, 15B2 and 15C, in step 1301, CICE is performed on silicon-on-x (e.g., silicon-on-insulator (SOI), silicon-on-sapphire (SOS), silicon-on-glass (SOG), etc.). For example, as illustrated in FIG. 14A, CICE is performed on the silicon-on-x structure, where silicon is represented by 1402 and “x” is represented by 1401. As shown in FIG. 14B, after CICE is performed, silicon 1402 has been etched forming nanostructures.

Referring to FIG. 14B, FIG. 15A illustrates the silicon core with stable I-beam-like structures. FIG. 15B1-15B2 illustrate a top view of the silicon core with 8 degrees of symmetry. FIG. 15C illustrates a top view of the axisymmetric silicon core. In one embodiment, the silicon core has a geometry designed to consider structural and performance constraints. In one embodiment, the silicon core is doped.

In optional step 1302, silicon 1402 is optionally oxidized.

In step 1303, active material 1403 is deposited on silicon 1402, such as the oxidized silicon, as shown in FIG. 14C. In one embodiment, active material 1403 includes one of the following: titanium dioxide, aluminum oxide, palladium, platinum, tungsten, titanium nitride, tantalum nitride, copper, SiNₓ, SnOₓ, and ZnOₓ.

In step 1304, active material 1403 is etched back as shown in FIG. 14D, which illustrates the final device.

An illustration of different variants of the nanostructure geometry using the conformal deposition process of FIG. 13 are shown in FIGS. 16A-16C in accordance with an embodiment of the present invention.

Referring to FIGS. 16A-16C in conjunction with FIG. 14B, FIGS. 16A-16C illustrate various top views of the nanostructure geometry variants of the silicon core.

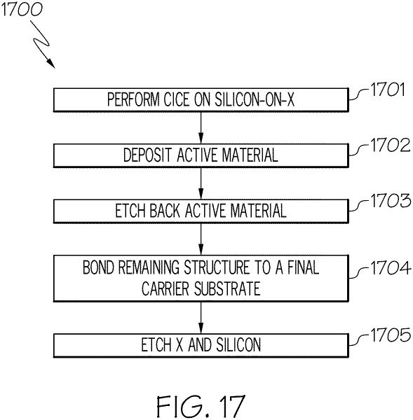

For certain applications, materials other than silicon are needed to improve performance. A replacement process is described in FIGS. 17, 18A-18D, 19 and 20A-20E to fabricate anisotropic high aspect ratio nanostructures with desired materials. In one embodiment, pillars in the active material may require holes to be etched in silicon. To prevent wandering, the holes could be connected. The connections could later be oxidized or filled-up using ALD, CVD, etc.

In one embodiment, material is deposited on silicon nanostructures etched using CICE using conformal deposition methods, such as atomic layer deposition (ALD), chemical vapor deposition (CVD), physical vapor deposition, thermal oxidation, electrodeposition, etc.

Deposited materials include metal oxides, metal nitrides, metals, semiconductors, insulators, etc., such as Al₂O₃, TiN, W, TiO₂, Pd, Pt, SiO₂, HfO₂, Cu etc., and are selected based on the desired device properties. Devices include metalenses, metamaterials, thermoelectrics, battery electrodes, gas sensors, etc.

After the material is deposited, the silicon nanostructures are removed, resulting in nanostructures of the opposite tone in the deposited material. In one embodiment, silicon nanostructures are removed by accessing the silicon and etching it using wet etching (tetramethylammonium hydroxide (TMAH), potassium hydroxide (KOH), ethylene di-amine pyro-catechol (EDP), etc.), plasma etching, dry etching (XeF₂), etc.

Access to silicon is created by (a) using silicon on a substrate - and removing the bond between the silicon and the substrate using wet etching (e.g., SOI wafer, where the oxide is etched away using hydrogen fluoride (HF)), (b) exfoliating the silicon to get thin silicon which is subsequently removed, and (c) etching all the silicon away from the back.

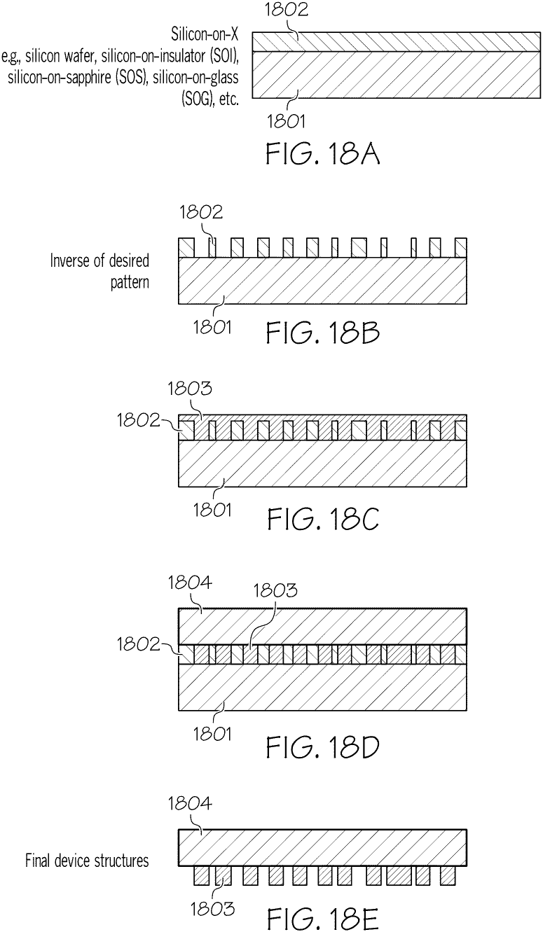

Referring now to FIG. 17, FIG. 17 is a method 1700 for obtaining the desired material high aspect ratio (HAR) nanostructures using a replacement process and atomic layer deposition (ALD) in accordance with an embodiment of the present invention. FIGS. 18A-18E depict cross-sectional views for obtaining the desired HAR nanostructures using the steps described in FIG. 17 in accordance with an embodiment of the present invention.

Referring to FIG. 17, in conjunction with FIGS. 18A-18E, in step 1701, CICE is performed on silicon-on-x (e.g., silicon wafer, silicon-on-insulator (SOI), silicon-on-sapphire (SOS), silicon-on-glass (SOG), etc.) as shown in FIGS. 18A-18B. For example, as illustrated in FIG. 18A, CICE is performed on the silicon-on-x structure, where silicon is represented by 1802 and “x” is represented by 1801. As shown in FIG. 18B, after CICE is performed, silicon 1802 has been etched forming nanostructures, which is the inverse of the desired pattern.

In step 1702, active material 1803 is deposited on the structure of FIG. 18B, including on the etched silicon 1802 and structure 1801 as shown in FIG. 18C. In one embodiment, active material 1803 includes one of the following: titanium dioxide, aluminum oxide, palladium, platinum, tungsten, titanium nitride, tantalum nitride, copper, SiNₓ, SnOₓ, and ZnO_{X}.

In step 1703, active material 1803 is etched back as shown in FIG. 18D.

In step 1704, after etching back active material 1803, the remaining structure is bonded to a final carrier substrate 1804 (e.g., glass) as shown in FIG. 18D.

In step 1705, structure 1801 and silicon 1802 are etched, such as via hydrogen fluoride (HF) for structure 1801 if structure 1801 is glass and via KOH for silicon 1802, as shown in FIG. 18E, which is the final device structure. In one embodiment, structure 1801 and silicon 1802 are removed using a wet etchant, a dry etchant or plasma etching.

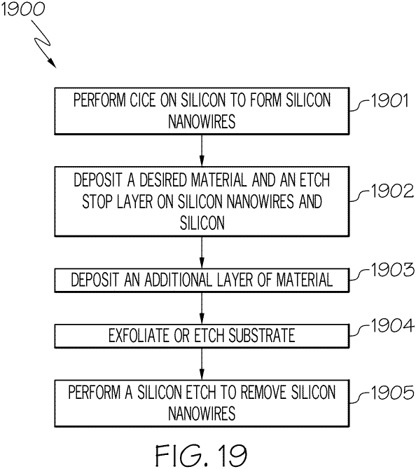

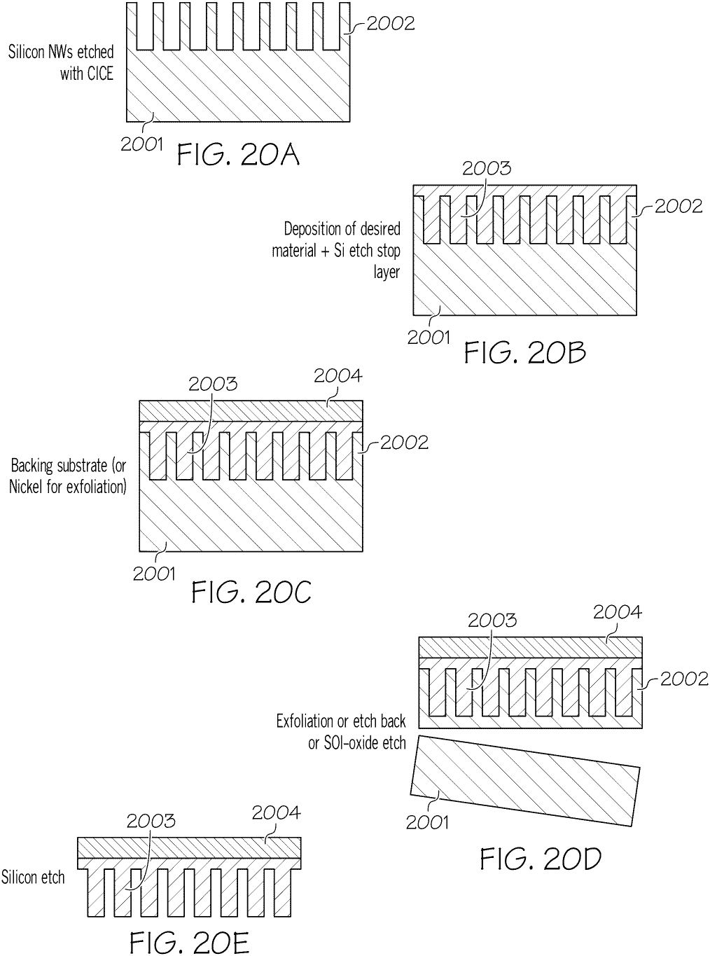

FIG. 19 is a flowchart of a method 1900 for obtaining the desired material high aspect ratio (HAR) nanostructures using a replacement process and atomic layer deposition (ALD) after exfoliation of silicon in accordance with an embodiment of the present invention. FIGS. 20A-20E depict cross-sectional views for obtaining the desired HAR nanostructures using the steps described in FIG. 19 in accordance with an embodiment of the present invention.

Referring to FIG. 19, in conjunction with FIGS. 20A-20E, in step 1901, CICE is performed on silicon 2001 (e.g., SOI substrate) to form silicon nanowires 2002 as shown in FIG. 20A.

In step 1902, a desired material and an etch stop layer 2003 are deposited on silicon nanowires 2002 and silicon 2001 as shown in FIG. 20B. In one embodiment, the etch stop layer is used to stop the etching process.

In step 1903, an additional layer of material (e.g., nickel) 2004 is deposited on layer 2003 as shown in FIG. 20C.

In step 1904, substrate 2001 is exfoliated or etched back (e.g., oxide etch) as shown in FIG. 20D.

In step 1905, a silicon etch is performed to remove silicon nanowires 2002 as shown in FIG. 20E. In one embodiment, silicon nanowires 2002 are removed using a wet etchant, a dry etchant or plasma etching.

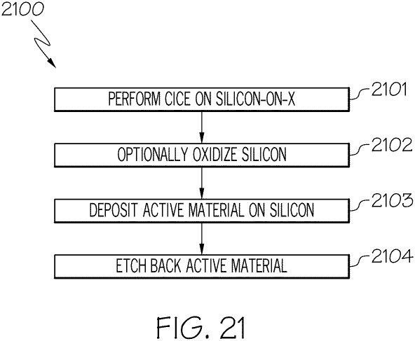

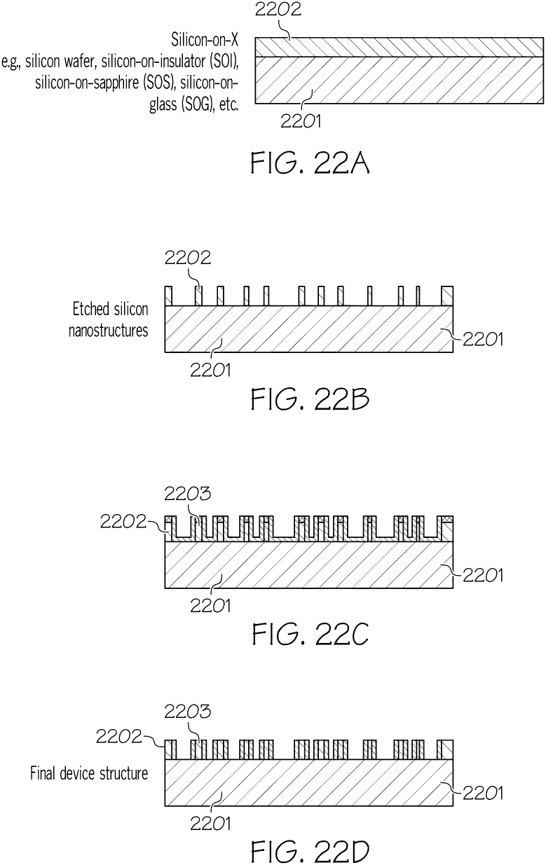

Alternatively, the silicon can remain unetched in applications where removal does not improve device properties. In one embodiment, the silicon is oxidized before deposition of the desired material. FIG. 21 is a flowchart of a method 2100 for achieving nanostructures in the desired material in accordance with an embodiment of the present invention. FIGS. 22A-22D depict cross-sectional views achieving nanostructures in the desired material using the steps described in FIG. 21 in accordance with an embodiment of the present invention.

Referring to FIG. 21, in conjunction with FIGS. 22A-22D, in step 2101, CICE is performed on silicon-on-x (e.g., silicon wafer, silicon-on-insulator (SOI), silicon-on-sapphire (SOS), silicon-on-glass (SOG), etc.) as shown in FIGS. 22A-22B. For example, as illustrated in FIGS. 22A-22B, CICE is performed on the silicon-on-x structure as shown in FIG. 22A, where silicon is represented by 2202 and “x” is represented by 2201, resulting in etched silicon nanostructures as shown in FIG. 22B.

In optional step 2102, silicon 2202 is optionally oxidized.

In step 2103, active material 2203 is deposited on silicon 2202, such as the oxidized silicon, as shown in FIG. 22C.

In step 2104, active material 2203 is etched back as shown in FIG. 22D, which illustrates the final device.

In one embodiment, silicon nanostructures are etched using CICE in a tool made for uniform, high throughput CICE process, and the nanostructures are subsequently oxidized using anodic oxidation in the same tool with the desired electrolyte for oxidation.

The principles of the present invention perform CICE for particle separation using deterministic lateral displacement (DLD).

Detection of small concentrations of biomolecules can enable early detection of diseases and to monitor the patient response to treatment. Such diagnostic tools can inform crucial decisions regarding the treatment method and improve the treatment outcome of the patient. At early stages of disease, the concentration of disease markers is very low and hard to detect in the typical specimen, such as blood, urine, blood plasma, serum, etc. Capturing and separating biomarkers, such as tumor cells and exosomes, can enable sensors to detect them. High density arrays of vertical nanowires show high capture efficiency and yield at high throughput. The geometry of the nanowire arrays can be tuned to capture biomolecules of desired sizes.

Deterministic lateral displacement (DLD) is a microfluidic technology which precisely separates particles in a fluid medium, larger and smaller than a critical size using specific arrangements of pillars in arrays placed within a microfluidic channel. The gaps between the pillars and the placement of the pillars in an array determine the critical particle size and separation pathways. Particles smaller than a critical size follow zigzag motion, and particles larger than the critical size follow bumping modes.

The effect of different post shapes has been investigated in the microscale. Circular posts have zones at the top of the post where the flow velocity is zero, leading to particle clogging and deformation of soft particles. Triangular, streamlined (airfoil shaped), I-shaped, diamond and quadrilateral posts have been investigated with the aim of reducing resistance within the device, increasing flow rates at low pressure heads, and directing the motion of irregular and/or deformable particles in a fluid medium and to increase their effective diameter.

At the microscale, flow is predominantly laminar, and any mixing happens by diffusion. Such diffusion may reduce the separation efficiency if the flow velocity is below a certain range, determined by the Peclet number (Pe) – the ratio of diffusion time to convection time. For small particles, (<10 micrometers in size), the diffusion time, and therefore the Peclet number is lower, which may lead to more prominent diffusive effects that can reduce their separation efficiency. For smaller particles, the gap between pillars is lower which causes a reduction in flow rates and particle times for a given fluid pressure. Soft particles may deform due to shear stress between the pillars. An effective size should be considered for designing the pillar arrays instead of the actual size. Irregularly shaped particles flowing in a DLD tend to orient themselves such that their smallest dimension is the critical dimension. In one embodiment, very shallow constrictions are used to limit the range of possible orientations, but they reduce the flow rates and increase flow separation times. Reducing the post gap using nanolithography instead of reducing the gap with microscale pillars can achieve the same separation rates with greater throughput.

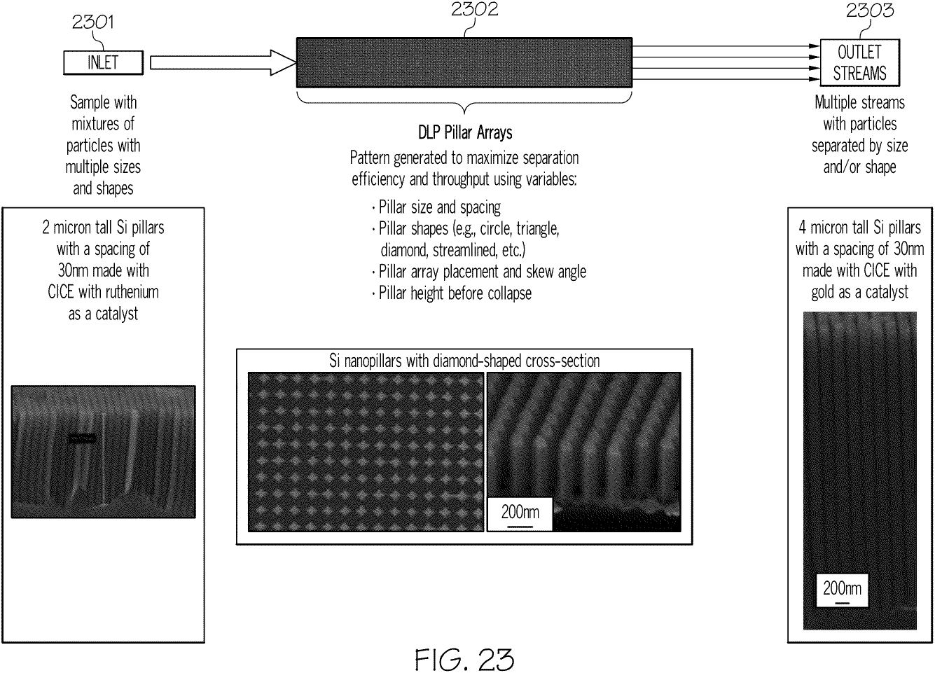

Taller posts lead to a higher throughput, limited by the aspect ratio before collapse. Materials used in posts are important – polydimethylsiloxane (PDMS) posts cause cell adhesion and deform considerably under pressure. Silicon pillars are more robust. However, fabrication of HAR silicon pillars with small gaps using plasma etching leads to etch taper, which changes the gap size. Catalyst influenced chemical etching (CICE) can be used to make HAR silicon pillars with small gaps, as well as sharp cross-sections, as shown in FIG. 23. FIG. 23 illustrates silicon nanopillars made with CICE for DLD-based particle separation in accordance with an embodiment of the present invention. The aspect ratios for pillars with small gaps can be optimized using analog-CICE to determine critical collapse heights experimentally. Nanopillars of optimized shapes, sizes and pillar array spacings can be tested using analog metal assisted chemical etching (MACE). In one embodiment, the catalysts for CICE can be Ru, Pd, Pt, Au, Ag, etc.

In one embodiment, the throughput of particle separation in a fluid medium is increased by designing pillars such that their height is maximized without causing substantial collapse to maximize the throughput of the fluid through the structures without increasing the spacing between the structures. The height of the nanostructure array is defined by the maximum height before substantial collapse to maximize the aspect ratio of the nanostructure array.

The spacing is defined by the critical particle size to be separated. In one embodiment, the pillar size is determined by optimizing the maximum collapse height and minimum pillar size to increase flow rates between the pillars.

In one embodiment, the spacing or gap between the pillars is less than 100 nm. In another embodiment, the spacing is less than 200 nm. In one embodiment, the spacing is less than 50 nm. In one embodiment, the spacing is less than 25 nm. The aspect ratios of the pillars can vary from greater than 5, greater than 10, and greater than 20. In one embodiment, the aspect ratio of the pillars is greater than 50. The aspect ratio is defined as the ratio between the height of the pillars and the critical feature size of the pillar cross-section.

In one embodiment, the nanopillars fabricated using CICE have a critical dimension of less than 200 nm, a height of more than 200 nm, and a wall taper angle greater than 89.5 degrees. In another embodiment, the nanopillars have a cross-section geometry having sharp corners, with a corner radius less than 5 nm. As shown in FIG. 23, the inlet 2301 (sample with mixtures of particles with multiple sizes and shapes) is inputted to DLD pillar arrays 2302 forming outlet streams 2303 (multiple streams with particles separated by size and/or shape). DLD pillar arrays 2302 include patterns generated to maximize separation efficiency and throughput using one or more of the following variables: pillar size and spacing; pillar shapes (e.g., circle, triangle, diamond, streamlined, etc.); pillar array placement and skew angle; and pillar height before collapse. FIG. 23 shows an example of diamond-shaped silicon nanopillars having a critical dimension of less than 130 nm, pitch of 200 nm and a corner radius of the diamond tips less than 5 nm.

The principles of the present invention may also utilize CICE for sensors.

Detection of biomarkers has been demonstrated with silicon nanowire devices functionalized with biomolecules, such as nucleic acids, antibodies, aptamers, etc. Detection ranges of aM-nM for nanowire FETs, and aM-fM for nanowire memristor sensors have been reported.

However, the nanowires used in devices (both for capture of desired biomarkers as well as in sensors to detect low concentrations of biomarkers) are expensive to fabricate and suffer from variations in device performance. Plasma etching of the nanowires causes rough surfaces and non-vertical sidewalls, which reduces the capture efficiency. Fabrication involves expensive and non-scalable processes, such as e-beam lithography and/or nanowire transfer with precise alignment.

SiNW field-effect transistor (FET) sensors are patterned using e-beam lithography and etched by plasma etching. Increasing the aspect ratio of the nanowire (e.g., by making it a finFETs) may improve sensitivity. CICE can be used to etch tall fins with no etch taper to avoid device-to-device variation and improve signal-to-noise ratio.

Fabrication of memristor sensors requires highly controlled plasma etch and oxidation. Alternatively, CICE can be used to fabricate multilayers of horizontal nanowires, similar to the fabrication of nanosheet FETs, using silicon superlattice etching. A description regarding using CICE to fabricate multilayers of horizontal nanowires is provided in U.S. Pat. Application Publication No. 2020/0365464), which is incorporated by referenced herein in its entirety.

This results in fins with alternating layers of non-porous and porous silicon layers. The porous silicon layers can be removed in sensing areas, and the drain and source areas are defined by depositing and annealing a metal, such as nickel to get nickel silicide. This method enables inexpensive, precise large-scale fabrication of highly sensitive silicon nanowire based memristor sensors as well as other types of sensors, such as a transistor-based sensor, a resistance-based sensor, a capacitance-based sensor and a fluorescence-based sensor.

Additionally, the principles of the present invention enable self-aligned imprint lithography (SAIL) for low-cost lithography.

Patterning of sensing elements (such as nanowires, fins, etc. for transistors and suspended nanowires for memristors) can be done along with patterning of sources, drains, gates, metal lines, and transducer circuits using self-aligned imprint lithography. This reduces or eliminates overlay errors and cost of multiple lithography steps. In one embodiment, a multitier template with the required features is used for a single step lithography, with each tier of the template used for a particular etch or deposition step to create sensors. The next patterning step is avoided by using etching to move to the next tier of the already imprinted resist features.

Furthermore, the principles of the present invention enable packing of devices made with CICE.

High aspect ratio nanostructures made using CICE for various applications are post-processed and packaged to prevent collapse and improve mechanical and chemical stability with minimal effect on the performance of the devices.

For metalens applications, the interstitial space in the core-shell structure (shown in FIGS. 22A-22D and 23) can be filled with a transparent material that acts as a protectant against mechanical and chemical damage, and potentially against nanostructure collapse as well (in applications where the nanostructures might be subjected to high accelerations). This material could be one or more polymer coatings (one of the coating layers, for instance, could be a thin coat of a fluoropolymer to make the device surface hydrophobic and resistant to damage due to moisture, while retaining transparency), and transparent insulating oxide and nitride films, such as SiO₂, Al₂O₃, Si₃N₄, etc. In another embodiment, a transparent plate could be used as a cover, and the space between the transparent plate and the core-shell structures could be filled with a fluid, such as air, water, etc.

Deposition techniques, such as glancing angle deposition (GLAD), ALD, CVD, etc. could be used for the deposition of the transparent insulating oxide and nitride films. Additionally, the coating layer adjacent to the metalens nanostructures could be an ultra-low refractive index material. This could be integrated into the metalens design, without adversely affecting the optical characteristics of either, using a co-optimization of the metalens and the low-index material. Further, the core-shell structure can also be covered with a plate made of a transparent material that acts as an additional protectant against mechanical and chemical damage.

For nanopillar arrays made for DLD applications, cover plates can be used to seal the device. Precise bubble-free bonding of a top cover on the nanopillar arrays is required to restrict motion of particles in fluid to be separated. This can be done using actuators to bring down a top cover (that is machined to have through holes for fluid inlets and outlets) precisely using multiple voice coil actuators. Additionally, to improve throughput, multiple chips with pillar arrays can be stacked and bonded together. In one embodiment, a conformal film (e.g., polymer materials, such as polycarbonate (PC), or softer materials, such as polydimethylsiloxane (PDMS), etc.) is bonded to the top of the pillar arrays, followed by a rigid cover plate, such as glass that is sufficiently transparent and conformal (thickness of <0.7 mm).

Batteries with nanostructured electrodes are assembled with the desired electrolyte, anode, and cathode. Nanostructured thermoelectric devices are packaged to include electrical connections to the nanowire arrays. Sensors are packaged to include electrical circuitry and have the sensing elements exposed for detection of analytes.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method for fabricating silicon nanostructures, the method comprising:
depositing an etch uniformity improving layer on a substrate;
depositing a catalyst on said substrate or said etch uniformity improving layer, wherein said catalyst layer is contacting a portion of said substrate or said etch uniformity improving layer; and
exposing said catalyst as well as said substrate or said etch uniformity improving layer to an etchant, wherein said catalyst causes etching of said substrate thereby creating etched nanostructures.

2. The method as recited in claim 1, wherein said catalyst comprises one or more of the following: Au, Pt, Pd, Mo, Ru, Ir, Ag, Cu, Ni, W, TiN, TaN, RuO₂, IrO₂, graphene, Ti, and carbon.

3. The method as recited in claim 1, wherein said etch uniformity layer comprises a material that gets etched in an influenced chemical etching (CICE) etchant.

4. The method as recited in claim 1, wherein said etch uniformity layer is thermally grown silicon oxide having a thickness greater than 5 nm or is a native silicon oxide layer.

5. A method for fabricating silicon nanostructures, the method comprising:
depositing an etch uniformity improving layer on a substrate;
depositing and pattering a resist forming a resist layer with a plurality of features, wherein said resist layer includes a residual layer of thickness less than 100 nm;
etching said resist layer to remove said residual layer;
depositing a catalyst on said substrate or said etch uniformity improving layer, wherein said catalyst is contacting a portion of said substrate or said etch uniformity improving layer; and
exposing said catalyst as well as said substrate or said etch uniformity improving layer to an etchant, wherein said catalyst causes etching of said substrate thereby creating etched nanostructures.

6. The method as recited in claim 5, wherein said catalyst comprises one or more of the following: Au, Pt, Pd, Mo, Ru, Ir, Ag, Cu, Ni, W, TiN, TaN, RuO₂, IrO₂, graphene, Ti, and carbon.

7. The method as recited in claim 5, wherein said etch uniformity layer comprises a material that gets etched in an influenced chemical etching (CICE) etchant.

8. The method as recited in claim 5, wherein said etch uniformity layer is thermally grown silicon oxide having a thickness greater than 5 nm or is a native silicon oxide layer.

9. A method for fabricating nanostructures of varying heights, the method comprising:
providing a catalyst layer on a surface of a semiconducting substrate, wherein said catalyst layer comprises a plurality of features and one or more intentional discontinuities; and
exposing said catalyst layer on said surface of said semiconducting substrate to an etchant, wherein said catalyst layer causes etching of said semiconducting substrate starting from said one or more intentional discontinuities, wherein fabricated structures have a height variation with features closest to said one or more intentional discontinuities having a maximum height.

10. The method as recited in claim 9, wherein said catalyst layer comprises one or more of the following: Au, Pt, Pd, Mo, Ru, Ir, Ag, Cu, Ni, W, TiN, TaN, RuO₂, IrO₂, graphene, Ti, and carbon.

11. The method as recited in claim 9, wherein said one or more intentional discontinuities are created using one or more the following: focused ion beam, photolithography, imprint lithography, laser writing, and pattern geometries.

12. The method as recited in claim 9, wherein a shape of said one or more intentional discontinuities comprises one of the following: a circular pinhole, a line, and a series of intersecting lines.

13. The method as recited in claim 9, wherein a gradient of etch depth variation is determined by patterning of said one or more intentional discontinuities and etchant concentrations and diffusion.

14. A method for fabricating silicon nanostructures, the method comprising:
patterning a polymer resist on a substrate with a plurality of features;
depositing a material conformally on said polymer resist to reduce spacing between said plurality of features;
providing a catalyst layer on said substrate, wherein said catalyst layer is patterned using said plurality of features with said reduced spacing such that said catalyst layer contacts only a portion of said substrate; and
exposing said catalyst layer to an etchant, wherein said catalyst layer causes etching of said substrate thereby creating etched nanostructures.

15. The method as recited in claim 14, wherein said catalyst layer comprises one or more of the following: Au, Pt, Pd, Mo, Ru, Ir, Ag, Cu, Ni, W, TiN, TaN, RuO₂, IrO₂, graphene, Ti, and carbon.

16. The method as recited in claim 14, wherein said conformal material is deposited using one of the following: atomic layer deposition, chemical vapor deposition, and physical vapor deposition.

17. The method as recited in claim 14, wherein said conformal material is one or more of the following: a fluorocarbon, silicon dioxide, aluminum oxide and titanium nitride.

18. A method for fabricating nanostructures in a material, the method comprising:
etching silicon structures using catalyst influenced chemical etching, wherein said etched silicon structures are designed to avoid substantial collapse;
depositing one or more materials conformally on said etched silicon structures; and
creating access to said etched silicon structures and removing said etched silicon structures selectively leaving said one or more materials substantially the same.

19. The method as recited in claim 18, wherein said catalyst influenced chemical etching exposes a patterned catalyst on a semiconductor substrate to an etchant, wherein said patterned catalyst layer causes etching of said semiconductor substrate.

20. The method as recited in claim 18, wherein said patterned catalyst comprises one or more of the following: Au, Pt, Pd, Mo, Ru, Ir, Ag, Cu, Ni, W, TiN, TaN, RuO₂, IrO₂, graphene, Ti, and carbon.

21. The method as recited in claim 18, wherein said one or more deposited materials are one or more of the following: titanium dioxide, aluminum oxide, palladium, platinum, tungsten, titanium nitride, tantalum nitride, copper, SiNₓ, SnOₓ, and ZnOₓ.

22. The method as recited in claim 18, wherein access to said etched silicon structures is enabled by one of the following: bonding a top with a backing layer and removing silicon from a back of a silicon wafer, etching back a top of a deposited material and etching exposed silicon, using exfoliation to thin a top layer of a substrate before etching silicon from a back of said substrate, and using a silicon-on-insulator wafer and etching an insulator layer to lift-off a top patterned layer.

23. A method for fabricating nanostructures in a silicon layer on a non-silicon layer, the method comprising:
etching nanostructures in silicon using metal assisted chemical etching, wherein said etched nanostructures are designed to avoid substantial collapse; and
partially or completely oxidizing said etched nanostructures.

24. The method as recited in claim 23, wherein said non-silicon layer is one of the following: silicon oxide, sapphire, a polymer, and a metal.

25. The method as recited in claim 23, wherein said patterned catalyst layer comprises one or more of the following: Au, Pt, Pd, Mo, Ru, Ir, Ag, Cu, Ni, W, TiN, TaN, RuO₂, IrO₂, graphene, Ti, and carbon.

26. Nanostructures in a silicon layer on a non-silicon layer that possess optical lensing properties, wherein a core geometry is first etched into said silicon layer while substantially avoiding collapse, wherein said core geometry is subsequently oxidized partially or fully.

27. The nanostructures as recited in claim 26, wherein said core geometry is first etched into said silicon layer using catalyst influenced chemical etching.

28. The nanostructures as recited in claim 26, wherein a shell material is deposited on said core geometry.

29. The nanostructures as recited in claim 28, wherein said shell material comprises one of the following: titanium dioxide and silicon dioxide.

30. The nanostructures as recited in claim 26, wherein a nanostructure wall angle of one of said nanostructures is greater than 89.5 degrees at all points on a side wall except for a top and a bottom of said side wall.

31. The nanostructures as recited in claim 26, wherein a core structure of one or more of said nanostructures contains anti-reflective structures.

32. The nanostructures as recited in claim 26, wherein a shell structure of one or more of said nanostructures contains anti-reflective structures.

33. A device using silicon nanostructures, the device comprising:
silicon nanostructures designed to separate particles in a fluid medium having different size, shape or flow properties in a nanostructure array, wherein spacing between at least a pair of silicon nanostructures is less than 50 nm, wherein a nanostructure wall angle of one or more of said silicon nanostructures is greater than 89.5 degrees at all points on a side wall except for a top and a bottom of said side wall.

34. The device as recited in claim 33, wherein an aspect ratio of said silicon nanostructures is greater than 10.

35. The device as recited in claim 33, wherein said silicon nanostructures comprise:
pillars with nanoshape cross-sectional geometries having cross-sections with sharp corners whose radius of curvature < 10 nm.

36. The device as recited in claim 33, wherein said silicon nanostructures are fabricated using catalyst influenced chemical etching.

37. The device as recited in claim 33, wherein said nanostructure array is designed to separate particles in a fluid medium having different size, shape or flow properties, wherein spacing in said nanostructure array is designed to separate said particles.

38. A device for separation and detection of biological species, the device comprising:
silicon nanostructures fabricated using catalyst influenced chemical etching, wherein said silicon nanostructures are designed for particle separation in a fluid medium; and
sensors which are used to detect target species in said separated particles, wherein said sensors generate electrical and/or optical signals based on desired target species detection.

39. The device as recited in claim 38, wherein said silicon nanostructures form a deterministic lateral displacement array for particle separation, wherein increasing a concentration of particles improves sensor signal-to-noise ratio.
